(19) United States
(12) Patent Application Publication
(10) Pub. No.: US 2026/0259120 A1
(43) Pub. Date: Sep. 3, 2026

(54) DEVICE AND METHOD FOR TESTING BOUNDARY PILLAR FAILURE INDUCED BY TAILINGS BACKFILL IN OPEN PIT

(71) Applicant: Northeastern University, Shenyang (CN)

(72) Inventors: Xiaowei GU, Shenyang (CN); Zhenguo ZHU, Shenyang (CN); Xiaochuan XU, Shenyang (CN); Xiaowei GE, Shenyang (CN); Fengdan WANG, Shenyang (CN); Qing WANG, Shenyang (CN); Hao WANG, Shenyang (CN)

(21) Appl. No.: 19/657,133

(22) Filed: Apr. 24, 2026

(30) Foreign Application Priority Data
Apr. 29, 2025 (CN) 202510549711.2

(51) Int. Cl. G01N 3/10; G01N 3/06

(52) U.S. Cl. CPC G01N 3/10; G01N 3/066; G01N 3/068; G01N 2203/0064; G01N 2203/0067; G01N 2203/0658

(57) ABSTRACT

The present disclosure relates to the technical field of boundary pillar failure induced by backfilling of open pits, and particularly to device and method for testing boundary pillar failure induced by tailings backfill in an open pit, comprising: a test assembly; a conveying assembly, connected to the test assembly and configured to uniformly feed tailing into the test assembly; a pressure control assembly, connected to the test assembly and configured to control pressure in the test assembly; and a monitoring assembly, disposed on the test assembly and configured to monitor data information within the test assembly when internal pressure of the test assembly is changed. The conveying assembly is configured to provide uniform distribution of tailings to the test assembly, the pressure control assembly is configured to simulate actual pressure conditions, and the monitoring assembly is configured to acquire data in real time.

### TECHNICAL FIELD

The present disclosure relates to the technical field of boundary pillar failure induced by backfilling of open pits, and particularly to a device and method for testing boundary pillar failure induced by tailings backfill in an open pit.

### BACKGROUND

As shallow mineral resources are gradually depleted, deep mining has become an inevitable trend. Concurrently, the growing emphasis on environmental protection and sustainable resource utilization has led to the gradual emergence of technology for backfilling open-pit mines with tailings for underground mining. On one hand, this technology can effectively reduce environmental pressure caused by tailings accumulation, lower safety risks such as tailings dam failure, avoid occupying large amounts of land resources, and mitigate damage to the surrounding ecological environment. On the other hand, reasonable tailings backfill can enhance the stability of open-pit slopes and reduce the costs of slope monitoring and remediation. Nevertheless, the technology for backfilling open-pit mines with tailings to underground mining is still in the experimental stage for the most part, and the selection of key technical parameters still requires a lot of experimental support. Among these, the thickness of the boundary pillar is a particularly critical parameter. As a key structure that separates different mining areas or protects important facilities, the stability of the boundary pillar is directly related to the overall safety of the mine. When tailings are backfilled into an open pit, the interaction between the backfill body and the surrounding rock, as well as stress variations during mining operations, can have complex effects on the stability of the boundary pillar. Without an accurate understanding of these influence mechanisms, the boundary pillar may become unstable and fail, potentially leading to a series of surface and underground safety accidents and posing a great threat to mine safety production.

Existing research in this field typically faces high experimental costs. This is primarily owing to the fact that conventional methods require large-scale experiments to simulate real mine environments. From site preparation and material collection to equipment investment, such experiments consume substantial resources. Moreover, the conduct of experiments in actual scenarios poses certain risks. For example, when simulating tailings backfill and mining-induced stress variations, sudden events such as boundary pillar instability may occur in an actual mine, posing a serious threat to the safety of both experimental staff and equipment. Therefore, there is an urgent need to provide a device and method for testing boundary pillar failure induced by tailings backfill in an open pit.

### SUMMARY

In view of the above shortcomings and deficiencies of the prior art, the present disclosure provides a device and method for testing boundary pillar failure induced by tailings backfill in an open pit. The present disclosure addresses the technical problems present in existing research on boundary pillar failure induced by tailings backfill in open pits, specifically the high costs and inherent risks associated therewith.

In order to achieve the above-mentioned objective, the main technical solution adopted by the present disclosure includes:

In a first aspect, embodiments of the present disclosure provide a device for testing boundary pillar failure induced by tailings backfill in an open pit.

In accordance with an embodiment of the present disclosure, there is provided a device for testing boundary pillar failure induced by tailings backfill in an open pit, including:
a test assembly;
a conveying assembly, connected to the test assembly and configured to uniformly feed tailings into the test assembly;
a pressure control assembly, connected to the test assembly and configured to control pressure in the test assembly; and
a monitoring assembly, disposed on the test assembly and configured to monitor data information within the test assembly when internal pressure of the test assembly is changed.

In some embodiments, the test assembly includes:
a test box;
a test plate, arranged in an interior of the test box, and configured to form a funnel-shaped open pit model in the test box;
a boundary pillar model, arranged in the test box and positioned at a bottom of the open pit model; and
a mining body component model, arranged in the test box and in contact with a bottom of the boundary pillar model, and configured to support the boundary pillar model.

In some embodiments, the mining body component model includes:
multiple first mining bodies, arranged in parallel in the test box and in contact with a bottom of the boundary pillar model;
second mining bodies, arranged between the adjacent first mining bodies and in contact with a bottom of the boundary pillar model.

In some embodiments, the conveying assembly includes:
a protective housing, arranged on the test box and formed at a bottom thereof with multiple discharge openings in communication with the open pit model;
a drive shaft, coaxially and rotatably mounted inside the protective housing;
a blocking plate, fixedly arranged on the drive shaft in an alternating manner, wherein the blocking plate blocks the discharge openings when the blocking plate is in contact with the discharge openings;
a material storage assembly, in communication with the protective housing, and configured to convey tailings into the protective housing; and
a vibration component, arranged on an outer wall of the test box, and configured to uniformly spread the tailings within the open pit model.

In some embodiments, the material storage assembly includes:
a storage bin, configured to store the tailings;
a delivery pipe, in communication between the storage bin and the protective housing; and
an auger, arranged in the delivery pipe and configured to convey tailings.

In some embodiments, the monitoring assembly includes:
a fibre grating sensor, arranged in an interior of the boundary pillar model;
a pressure sensor, arranged at a bottom of the mining body component model;
multiple acoustic emission probes, arranged at one side of the test box at equidistant intervals; and
thermal infrared cameras, arranged on the other side of the test box at equidistant intervals.

In some embodiments, the pressure control assembly includes:
a hydraulic cylinder; and
a pressure plate, arranged on a top of the test box and connected to an output end of the hydraulic cylinder, and configured to vary pressure in the open pit model.

In a second aspect, embodiments of the present disclosure provide a method for testing boundary pillar failure induced by tailings backfill in an open pit.

In accordance with an embodiment of the present disclosure, there is provided a method for testing boundary pillar failure induced by tailings backfill in an open pit, employing the device for testing boundary pillar failure induced by tailings backfill in an open pit according to the first aspect:
making the mining body component model, the boundary pillar model, and the open pit model successively arranged, wherein the mining body component model includes the first mining bodies and the second mining bodies arranged adjacent to each other;
uniformly laying and compacting tailings in the open pit model with a compaction degree of greater than or equal to 90% to form a filling model;
applying a vertical load to the filling model to obtain a compacted backfill model;
removing the first mining body and filling the removed part with a filling body, and thereafter removing the second mining body; if the boundary pillar model does not fail after mining, applying a static load to the backfill model at a rate of 0.1 MPa/min until the boundary pillar model fails; and simultaneously, acquiring historical data of the boundary pillar model and preprocessing the historical data to obtain processed data;
inputting the processed data into a hybrid deep learning model to obtain a trained hybrid deep learning model;
inputting real-time data into the trained hybrid deep learning model to obtain a safety factor of the pillar;
when the safety factor of the pillar is greater than or equal to 0.8 and less than 1.0, reducing a loading rate to 0.05 MPa/min;
when the safety factor of the pillar is greater than or equal to 0.5 and less than 0.8, suspending the static load applied to the backfill model; and
when the safety factor of the pillar is less than 0.5, stopping the static load applied to the backfill model, triggering an alarm, and saving experimental data.

In some embodiments, steps of making the open pit model and the boundary pillar model further include:
mixing barite powder, river sand, gypsum, and water in a ratio of 1:4:1.25:1.25, casting the mixture to form an open pit model at a scale of 1:100 or 1:200, and curing the model for 7 days;
mixing barite powder, river sand, gypsum, and water in a ratio of 1:3.75:1.5:1.25 to form the boundary pillar model, curing the boundary pillar model for 7 days, and then embedding the boundary pillar model into the open pit model at a predetermined position.

In some embodiments, the step of making the filling model further includes:
mixing tailings, cement, and water according to a mass ratio, wherein a cement content is selected from 5%, 10%, and 15%, a water-to-solid ratio is 0.2, and the tailings have a particle diameter of less than 2 mm.

The beneficial effects of the present disclosure are that the device and method for testing boundary pillar failure induced by tailings backfill in an open pit include a test assembly, a conveying assembly, and a pressure control assembly. The conveying assembly is configured to provide uniform distribution of tailings to the test assembly, the pressure control assembly is configured to simulate actual pressure conditions, and the monitoring assembly is configured to acquire data in real time. These assemblies together form a complete and effective experimental device. The present disclosure allows simulation experiments of boundary pillar failure induced by tailings backfill in open pit to be performed in a controllable and safe laboratory environment, thus solving the problems of high experimental costs and high risks inherent in conventional research.

### BRIEF DESCRIPTION OF THE DRAWINGS

FIG. 1 is a schematic perspective view of a device for testing boundary pillar failure induced by tailings backfill in an open pit according to the present disclosure;

FIG. 2 is a schematic cross-sectional view of a test assembly according to the present disclosure;

FIG. 3 is a schematic top view of a test assembly according to the present disclosure;

FIG. 4 is a schematic cross-sectional view of a conveying assembly according to the present disclosure;

FIG. 5 is a schematic cross-sectional view of a material storage assembly according to the present disclosure;

FIG. 6 is a schematic flow diagram of a test method for testing boundary pillar failure induced by tailings backfill in an open pit according to the present disclosure.

### REFERENCE NUMERALS IN FIGURES

100—a test assembly, 200—a conveying assembly, 300—a pressure control assembly, 400—a monitoring assembly;
110—a test box, 120—a test plate, 130—a boundary pillar model, 140—a mining body component model;
210—a protective housing, 220—a drive shaft, 230—a blocking plate, 240—a material storage assembly, 250—a vibration component;
310—a hydraulic cylinder, 320—a pressure plate;
410—a fibre grating sensor, 420—a pressure sensor, 430—an acoustic emission probe, 440—a thermal infrared camera;
141—a first mining body, 142—a second mining body;
241—a storage bin, 242—a delivery pipe, 243—an auger;
101—an open pit model, 201—a discharge opening.

### DETAILED DESCRIPTION

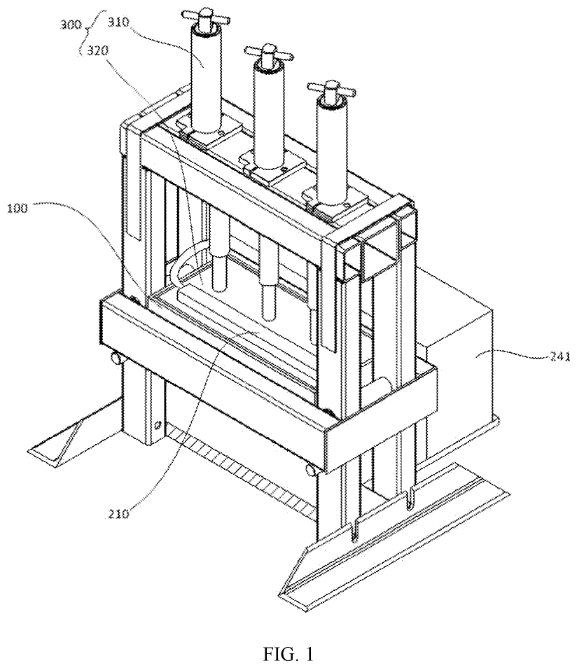

In order to better explain the present disclosure and facilitate understanding, a detailed description of the present disclosure is provided below with reference to the accompanying drawings and through specific embodiments. In the present document, directional terms such as ‘upper’, ‘lower’, etc., are based on the orientation shown in FIG. 1.

The following description provides a more detailed explanation of exemplary embodiments of the present disclosure with reference to the accompanying drawings, to facilitate a better understanding of the technical solutions described above. It should be understood that, although the accompanying drawings illustrate exemplary embodiments of the present disclosure, the present disclosure may be embodied in various forms and is not limited to the embodiments described herein. Conversely, these embodiments are provided to enable a clearer and more thorough understanding of the present disclosure and to fully convey the scope of the present disclosure to those skilled in the art.

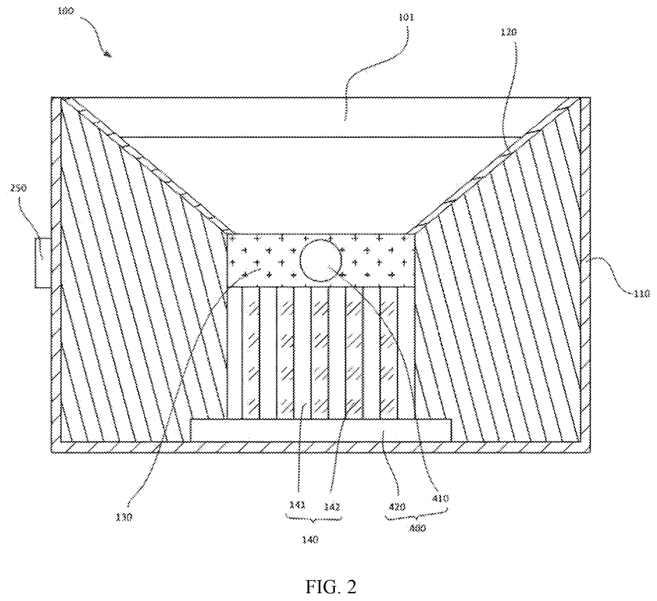

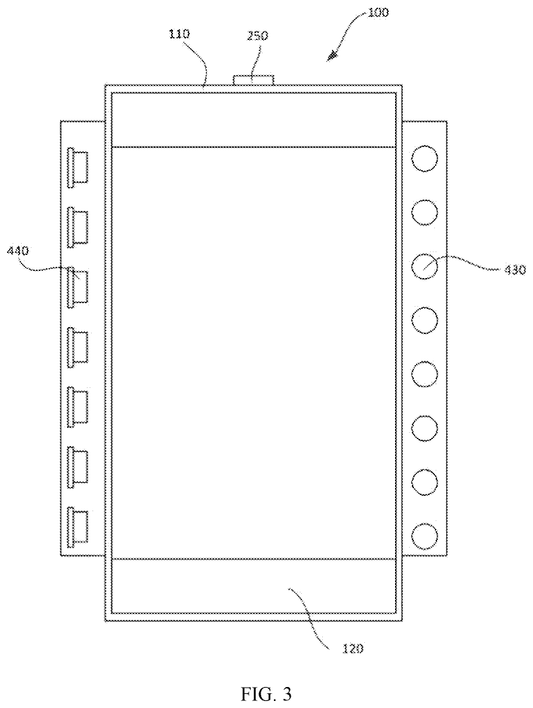

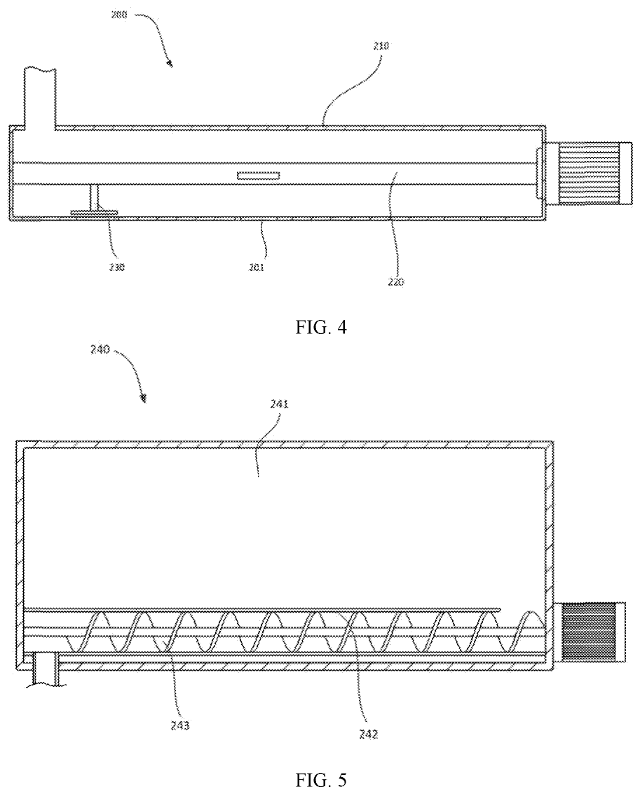

As shown in FIGS. 1-5, according to a first aspect of an embodiment of the present application, there is provided a device for testing boundary pillar failure induced by tailings backfill in an open pit, including: a test assembly 100; a conveying assembly 200, the conveying assembly 200 is connected to the test assembly 100 and configured to uniformly feed tailing into the test assembly 100; a pressure control assembly 300, the pressure control assembly 300 is connected to the test assembly 100 and configured to control pressure in the test assembly 100; a monitoring assembly 400, the monitoring assembly 400 is disposed on the test assembly 100 and configured to monitor data information within the test assembly 100 when internal pressure of the test assembly 100 is changed.

The device for testing boundary pillar failure induced by tailings backfill in an open pit provided in an embodiment of the present application includes a test assembly 100, a conveying assembly 200 and a pressure control assembly 300, wherein the test assembly 100 is configured to provide a basic space for simulating stress conditions of an open pit to be backfilled with tailings and a boundary pillar under the surrounding environment, and serves as a core component for the action of various experimental factors and data generation.

The conveying assembly 200 is connected to the test assembly 100, and is mainly used to uniformly feed tailings into the test assembly 100. This assembly is configured to ensure that the tailings can enter the test assembly 100 in a stable and uniform manner, and to simulate the distribution state of tailings during an actual backfilling process, thereby providing a basis for accurately simulating the interaction between tailings and the rock mass (boundary pillar) in subsequent processes. By controlling parameters of the conveying assembly 200, such as flow velocity and flow rate, the accumulation state of tailings under different backfilling conditions can be accurately simulated.

The pressure control assembly 300 is connected to the test assembly 100 and is responsible for controlling the pressure within the test assembly 100. In an actual mining process, pressure variation is one of the key factors affecting the stability of the boundary pillar. The pressure control assembly 300 is configured to simulate pressure changes under different mining stages and different working conditions by accurate control means, so that the pressure environment inside the test assembly 100 is made as close as possible to reality. Through such simulation, the influence of different pressure conditions on the stability of the boundary pillar can be deeply studied, which is of great significance for determining a reasonable mining solution and boundary pillar parameters.

The monitoring assembly 400 is disposed on the test assembly 100. When the internal pressure of the test assembly 100 is changed, real-time monitoring data information from the test assembly 100 can comprehensively reflect the data response of the boundary pillar during tailings backfilling and pressure variation. The monitoring assembly 400 is configured to obtain the stability variation of the boundary pillar under different experimental conditions by collecting and analyzing these data. In the experimental method, real-time data of the boundary pillar model 130 are collected in real time, and these data are preprocessed and then input into a hybrid deep learning model to obtain a safety factor of the pillar. According to the safety factor, different experimental operations are performed. The accurate monitoring provided by the monitoring assembly 400 supplies data support for the entire experiment, so that researchers can thoroughly understand the mechanical behavior of the boundary pillar during the experimental process and deeply analyze the inherent law of boundary pillar failure induced by tailings backfill in an open pit, which provides indispensable data support for the evaluation of boundary pillar stability.

In summary, all the assemblies are closely matched. The conveying assembly 200 is configured to provide uniformly distributed tailings for the test assembly 100, the pressure control assembly 300 is configured to simulate actual pressure conditions, and the monitoring assembly 400 is configured to acquire data in real time. Collectively, they constitute a complete and effective experimental device, enabling simulation experiments of boundary pillar failure induced by tailings backfill in an open pit to be performed in a controllable and safe laboratory environment, and solving the problems of high experimental costs and high risks inherent in conventional research.

As shown in FIGS. 1-5, in some examples, a test assembly 100 includes: a test box 110; a test plate 120, the test plate 120 is arranged in the interior of the test box 110, and is configured to form a funnel-shaped open pit model 101 in the test box 110; a boundary pillar model 130, the boundary pillar model 130 is arranged in the test box 110 and positioned at the bottom of the open pit model 101; and a mining body component model 140, the mining body component model 140 is arranged in the test box 110 and is in contact with the bottom of the boundary pillar model 130, and is configured to support the boundary pillar model 130.

In this technical solution, the test box 110 serves as an external frame structure of the test assembly 100, and is configured to accommodate and support various internal components, so as to provide a stable physical environment for the whole experiment, ensure that various components can operate in a relatively enclosed and stable space during the experiment, and avoid interference from external factors.

The test plate 120 is arranged in the interior of the test box 110, so that the funnel-shaped open-pit model 101 is formed in the test box 110. This funnel-shaped open-pit model 101 is used as part of the simulation of an actual open-pit shape. In actual open-pit mining activities, the shape and structure of the open pit have an important impact on tailings backfill and subsequent stress distribution conditions. By constructing the funnel-shaped open-pit model 101 in the test box 110, the experiment is enabled to more realistically simulate the natural accumulation of tailings in the open pit and the associated pressure distribution characteristics. Different shapes and sizes of the open pit may lead to different tailings accumulation patterns and stress transmission paths. The funnel-shaped open-pit model 101 is considered to be more consistent with typical open-pit designs. This allows conditions similar to those in the real world to be created for subsequent experiments, thereby enhancing the authenticity and reliability of the results.

The boundary pillar model 130 is arranged in the test box 110 and is positioned at the bottom of the open pit model 101. In actual mining, the boundary pillar model 130 is a key structure, the stability of the boundary pillar model 130 is directly related to the overall safety of the mine. In this experiment, the boundary pillar model 130 serves as the central component. Factors such as tailings backfill in the open pit model 101 and pressure variations can affect the boundary pillar model 130, thereby leading to changes in its mechanical state. By directly monitoring and studying the boundary pillar model 130, it may gain an in-depth understanding of the effects of various forces and stability changes experienced by the boundary pillar during tailings backfill in an open pit. This provides an important basis for assessing the stability of the boundary pillar and protecting it in actual mining operations.

The mining body component model 140 is arranged in the test box 110, and is in contact with the bottom of the boundary pillar model 130. The mining body component model 140 is mainly configured to support the boundary pillar model 130. In an actual mine environment, the mechanical interaction exists between the mining body and the boundary pillar. The mining body component model 140 is configured to simulate the structure and mechanical environment of a mining body in an actual mine. During the experimental process, the mining body component model 140 is operated according to experimental steps, so as to simulate the mining process of the actual mine.

For example, the spaces within the test box 110 that are located on both sides of the open pit model 101 are each configured as filled spaces, so as to simulate rock conditions in the field.

In summary, the various components of the test assembly 100 are closely matched. The test box 110 is configured to provide needed space, the test plate 120 is configured to construct the open pit model 101, the boundary pillar model 130 serves as the core object of study, and the mining body component model 140 is configured to simulate the mining environment. Such a design enables the test assembly 100 to accurately and effectively simulate the complex process of tailings backfill in an open pit and the influence of mining activities on boundary pillar stability as encountered in an actual mine, and provides a solid foundation for the entire test apparatus to realize its functions.

As shown in FIGS. 1-5, in some examples, the mining body component model 140 includes: multiple first mining bodies 141, the first mining bodies 141 are arranged in parallel in the test box 110 and in contact with the bottom of the boundary pillar model 130; second mining bodies 142, the second mining bodies 142 are arranged between the adjacent first mining bodies 141 and in contact with the bottom of the boundary pillar model 130.

In this technical solution, multiple first mining bodies 141 are provided. The first mining bodies 141 are arranged in parallel in the test box 110 and in contact with the boundary pillar model 130. This arrangement is used to simulate multiple mining units at different positions in actual mining. In the actual mining process, these scattered and parallel-arranged mining bodies gradually excavate the underground ore body, and each mining body may have a different degree of influence on the surrounding rock mass, including the boundary pillar. In the experimental environment, multiple parallel first mining bodies 141 are configured to truly reproduce the stress conditions associated with such multi-source mining, thereby providing simulation conditions for studying the influence of different degrees of mining activities on the boundary pillar.

The second mining bodies 142 are arranged between adjacent first mining bodies 141 and are also in contact with the bottom of the boundary pillar model 130. The second mining bodies 142 are arranged between the adjacent first mining bodies 141. In an actual mine, ore bodies in different areas may be mined in a specific order, which is determined based on geological conditions, mining plans, and other factors. By the arrangement of the second mining body 142, this complex mining condition is allowed to be simulated in the experiment. Contact of the second mining body 142 with the boundary pillar model 130 also transmits the forces generated by mining to the boundary pillar. Furthermore, because the second mining body 142 is located in a special position between the first mining bodies 141, the transmission path and distribution pattern of surrounding stresses are altered, thereby affecting the mechanical state of the boundary pillar.

As shown in FIGS. 1-5, in some examples, the conveying assembly 200 includes: a protective housing 210, the protective housing 210 is arranged on the test box 110 and formed at the bottom thereof with multiple discharge openings 201 in communication with the open pit model 101; a drive shaft 220, the drive shaft 220 is coaxially and rotatably mounted inside the protective housing 210; a blocking plate 230, the blocking plate 230 is fixedly arranged on the drive shaft 220 in an alternating manner, the blocking plate 230 blocks the discharge openings 201 when the blocking plate 230 is in contact with the discharge openings 201; a material storage assembly 240, the material storage assembly 240 is in communication with the protective housing 210, and the material storage assembly 240 is configured to convey tailings into the protective housing 210; and a vibration component 250, the vibration component 250 is arranged on the outer wall of the test box 110, the vibration component 250 is configured to uniformly spread the tailings within the open pit model 101.

In this technical solution, the protective housing 210 is mounted on the test box 110, and is configured to provide a protective and supporting structure for the internal components of the conveying assembly 200. The bottom part of the protective housing 210 is provided with multiple discharge openings 201, which are connected to the open pit model 101. The discharge openings 201 serve as channels through which tailings enter the open pit model 101 from the conveying assembly 200. The discharge openings 201 are arranged at equidistant intervals, which provides basic conditions for uniform laying of the tailings.

The drive shaft 220 is coaxially and rotatably mounted within the protective housing 210, and serves as a core component for power transmission during the entire conveying process. Driven by the external power source, the drive shaft 220 can rotate in a circular manner, and this rotation provides power for subsequent components connected to the drive shaft 220, thereby enabling them to work together to complete the tailings conveying operation.

The blocking plate 230 is fixed on the drive shaft 220 in an alternating manner and is arranged to cooperate with the discharge openings 201. When the blocking plate 230 is in contact with the discharge openings 201, the discharge openings 201 are completely blocked by the blocking plate 230, thereby preventing the tailings from falling. As the drive shaft 220 rotates, the blocking plate 230 is moved away from the discharge openings 201, allowing the tailings to fall into the open pit model 101 through the discharge openings 201. The alternating arrangement of the blocking plate 230 enables the tailings to be introduced from the protective housing 210 into the open pit model 101 in an intermittent and controllable manner, which helps to precisely control the flow rate and falling position of the tailings, thereby improving the distribution uniformity of the tailings within the open pit model 101.

The material storage assembly 240 is connected to the protective housing 210 and is configured to convey tailings to the protective housing 210. The material storage assembly 240 continuously and stably conveys tailings to the protective housing 210. The presence of the material storage assembly 240 ensures a sufficient supply of tailings during the conveying process and provides material support for the continuous simulation of the actual tailings backfilling process.

The vibration component 250 is arranged on the outer wall of the test box 110. For example, at least two vibration components 250 may be provided, and the at least two vibration components 250 are arranged on the outer wall of the test box 110 at equidistant intervals. The main function of the vibration component 250 is to uniformly spread the tailings within the open pit model 101. After the tailings enter the open pit model 101 through the discharge openings 201, gravity alone cannot guarantee a complete uniform distribution of the tailings. The vibration component 250 is configured to indirectly act on the tailings within the open pit model 101 by transmitting vibrational energy to the test box 110. Under the action of vibration, the tailings particles are subjected to additional external forces, which allow them to adjust their positions and gradually achieve a more uniform distribution state, ensuring that the compaction degree of the tailings is relatively consistent throughout the open pit model 101, thereby improving the authenticity and accuracy of the experimental simulation.

In summary, the conveying assembly 200 is configured to provide tailings through the coordination of various components. Specifically, tailings are supplied by the material storage assembly 240, the falling rhythm and position of the tailings are controlled by the drive shaft 220 and the blocking plate 230, and the uniform distribution of the tailings is assisted by the vibration component 250. In this manner, uniform feeding of tailings to the open pit model 101 within the test assembly 100 is achieved, which provides a good material laying foundation for studying the influence of tailings backfill on the boundary pillar in subsequent experiments.

As shown in FIGS. 1-5, in some examples, the material storage assembly 240 includes: a storage bin 241, the storage bin 241 is configured to store the tailings; a delivery pipe 242, the delivery pipe 242 is in communication between the storage bin 241 and the protective housing 210; and an auger 243, the auger 243 is arranged in the delivery pipe 242 and configured to convey tailings.

In this technical solution, the storage bin 241 is configured as a container for storing tailings. Its main function is to serve as a ‘warehouse’ that holds a certain amount of tailings to meet the demands of continuous conveying during the experiment. The conveying pipe 242 is configured as a ‘bridge’ connecting the storage bin 241 and the protective housing 210, and serves to establish communication between these two important components, thereby enabling directional conveying of the tailings. By connecting the storage bin 241 and the protective housing 210, a closed path is provided for the tailings to be transported from the storage area to the discharge area, and the tailings are prevented from being scattered or disturbed by external factors during the transportation process. For example, the inner wall of the conveying pipe 242 may be made of a material selected for smoothness and wear resistance. Such a material can reduce friction between the tailings and the inner wall of the pipe, to minimize wear, and to prolong the service life of the conveying pipe 242. A reliable connection method is employed to ensure that no shedding or leakage occurs during the transportation of the tailings and to ensure the sealing and stability of the entire conveying system.

The auger 243 is arranged in the interior of the conveying pipe 242, and is also referred to as a screw conveyor. The working principle of the auger 243 is as follows: a screw blade is rotated about a central axis, and during rotation, the tailings are pushed along the direction of the screw blade, so that the tailings are transported from the storage bin 241 to the protective housing 210. By this conveying method, the tailings can be transported from one end to the other end in a relatively smooth and efficient manner, and the conveying speed and flow rate of the tailings can be controlled by adjusting the rotational speed of the auger 243.

As shown in FIGS. 1-5, in some examples, the monitoring assembly 400 includes: a fibre grating sensor 410, the fibre grating sensor 410 is arranged in the interior of the boundary pillar model 130; a pressure sensor 420, the pressure sensor 420 is arranged at the bottom of the mining body component model 140; multiple acoustic emission probes 430, the acoustic emission probes 430 are arranged at one side of the test box 110 at equidistant intervals; and thermal infrared cameras 440, the thermal infrared cameras 440 are arranged on the other side of the test box 110 at equidistant intervals.

In this technical solution, the fibre grating sensor 410 is mounted inside the boundary pillar model 130. Under the influence of tailings backfill and mining activities, the internal mechanical state of the boundary pillar model 130 is changed in a complex manner, for example, the stress and strain distribution. The fibre grating sensor 410 is configured to accurately perceive changes in these internal physical quantities. Due to the sensitivity of the fibre grating to environmental parameters, when stress or strain changes inside the boundary pillar model 130, this causes a corresponding change in the reflection wavelength of the fibre grating. By detecting this wavelength variation, real-time information of the internal stress and strain of the boundary pillar model 130 can be obtained. This information is of great significance for understanding the internal stress state of the boundary pillar under different working conditions and for judging whether it is approaching a failure state. It is considered helpful for researchers to accurately grasp the process of stability change of the boundary pillar.

The pressure sensor 420 is positioned at the bottom of the mining body component model 140. During the mining simulation process, a pressure effect is exerted by the mining body component model 140 on the lower part. The role of the pressure sensor 420 is to accurately measure the magnitude and variation of this pressure. By obtaining pressure data of the pressure exerted by the mining body component model 140 on the bottom, the force applied by mining activities to the lower strata can be understood, which is of great value for studying how mining activity affects the boundary pillar through bottom transfer. Furthermore, when combined with other monitoring data, the mechanical transmission path and influence range of mining activities within the entire experimental system can be analyzed more comprehensively, thereby providing an important pressure parameter basis for further study of the relationship between tailings backfill and boundary pillar failure.

Multiple acoustic emission probes 430 are arranged on one side of the test box 110 at equidistant intervals. Acoustic emission is an elastic wave phenomenon generated during the deformation or fracture of a material. In the experiment, when the boundary pillar model 130 undergoes slight deformation, crack propagation, and eventual failure, acoustic emission signals are generated. The acoustic emission probes 430, being arranged at equidistant intervals, are configured to receive these acoustic emission signals over a wide range and to convert them into electrical signals for transmission and analysis. Through monitoring and analysis of the acoustic emission signals, information on the internal deformation and damage development of the material can be obtained by researchers, such as the determination of crack initiation location, propagation direction, and propagation rate.

The thermal infrared cameras 440 are arranged on the other side of the test box 110 at equidistant intervals. During the experiment, due to the influence of factors such as tailings backfill, mining activities, and force-induced deformation of the components, the surface temperature of various objects inside the test box 110 is changed. The thermal infrared cameras 440 are configured to convert received infrared radiation from the objects into thermal images, and to visually present the distribution of surface temperature of the objects and their variation over time. Monitoring by the thermal infrared cameras 440 can identify temperature anomaly areas during the experiment. These areas may be associated with phenomena such as stress concentration, energy dissipation, and damage development inside the objects. By analyzing the temperature changes, the distribution and conversion of energy during the experimental process can be further understood, and useful supplementary information is provided for the comprehensive study of the physical process of boundary pillar failure induced by tailings backfill in an open pit.

As shown in FIGS. 1-5, in some examples, the pressure control assembly 300 includes: a hydraulic cylinder 310; and a pressure plate 320, the pressure plate 320 is arranged on the top of the test box 110 and is connected to the output end of the hydraulic cylinder 310, and is configured to vary pressure in the open pit model 101.

In this technical solution, the hydraulic cylinder 310 serves as a power source for the pressure control assembly 300. A strong driving force is generated by the hydraulic cylinder 310 through pressure changes of hydraulic oil. In the experimental device, the piston rod of the hydraulic cylinder 310 is controlled to extend and retract, thereby providing an adjustable power output for the entire pressure control assembly 300. The hydraulic cylinder 310 is a core driving component for achieving pressure variations.

The pressure plate 320 is arranged at the top of the test box 110 and is closely connected to the output end of the hydraulic cylinder 310. The pressure plate 320 acts as a ‘pressure transmitter’, and the force generated by the hydraulic cylinder 310 is uniformly applied to the tailings within the open pit model 101 and to the model. The pressure plate 320 is positioned in the middle of the open pit model 101. For example, the length of the pressure plate 320 may be selected to be half of the top length of the open pit model 101, so that, when the pressure plate 320 contacts the top of the open pit model 101, the received pressure is ensured to be uniformly distributed over the surface of the open pit model 101, where the maximum pressure load is 1 MPa.

For example, the pressure plate 320 may be provided in a variety of different sizes and is optionally replaceable at the output end of the hydraulic cylinder 310.

When the pressure within the open pit model 101 is required to be changed during the experiment, the external hydraulic control system will adjust the hydraulic oil inside the hydraulic cylinder 310 according to preset parameters. For example, when the pressure of the hydraulic oil is increased, the piston rod of the hydraulic cylinder 310 is extended, and the extension of the piston rod drives the pressure plate 320 connected thereto to move downward. The pressure plate 320 gradually moves closer to the top of the open pit model 101 and exerts pressure thereon. This pressure is transmitted through the tailings to various parts of the open pit model 101, and thus, the internal pressure of the open pit model 101 is increased. Conversely, if the pressure is required to be decreased, the hydraulic control system can reduce the pressure of the hydraulic oil within the hydraulic cylinder 310, the piston rod is retracted, the pressure plate 320 is moved upward, and the pressure on the open pit model 101 is reduced. In this manner, the pressure control assembly 300 is enabled to accurately and continuously change the pressure within the open pit model 101, so as to meet the requirements of pressure simulation under different experimental conditions. The experimental results are closer to actual conditions, which facilitates an in-depth study of the relationship among tailings backfill, open pit pressure, and boundary pillar stability.

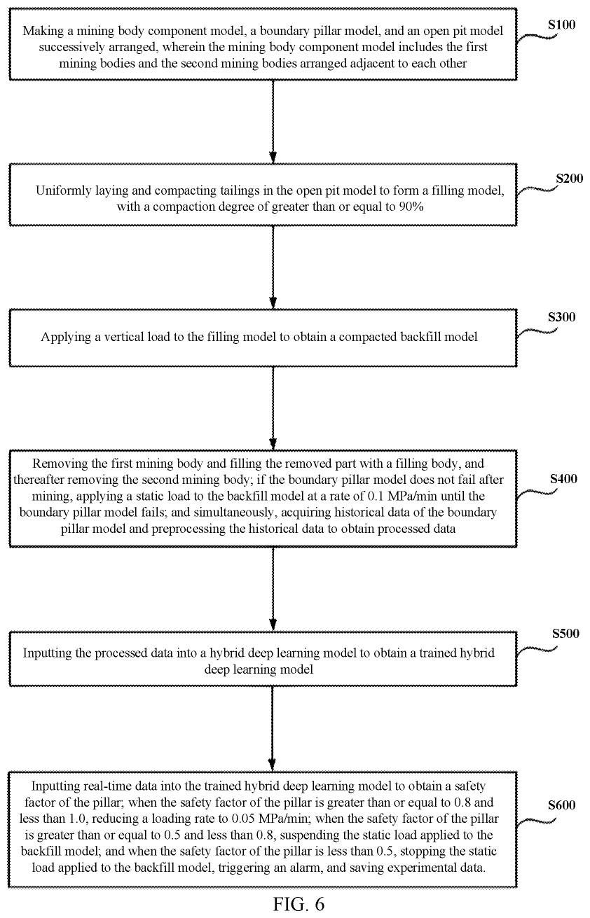

As shown in FIG. 6, a method for testing boundary pillar failure induced by tailings backfill in an open pit is provided according to the second aspect of the present application, employing the device for testing boundary pillar failure induced by tailings backfill in an open pit according to the first aspect:

S100: the mining body component model 140, the boundary pillar model 130, and the open pit model are made successively arranged, wherein the mining body component model 140 includes the first mining body 141 and the second mining body 142 arranged adjacent to each other.

In S100, steps of making the open pit model and the boundary pillar model further includes: the barite powder, river sand, gypsum, and water are mixed in a ratio of 1:4:1.25:1.25, the mixture is cast to form the open pit model 101 at a scale of 1:100 or 1:200, and the model is cured for 7 days; the barite powder, river sand, gypsum, and water are mixed in a ratio of 1:3.75:1.5:1.25 to form the boundary pillar model 130, the boundary pillar model 130 is cured for 7 days, and then the boundary pillar model 130 is embedded into the open pit model at a predetermined position.

S200: tailings are uniformly laid and compacted in the open pit model 101 to form a filling model, with a compaction degree of greater than or equal to 90%;

In S200, the step of forming a filling model further includes: a vibration screening machine is used and passed through a 10-mesh square-hole sieve to ensure that the particle size of the tailings is less than 2 mm, and the cementing material is prepared by mixing the tailings, cement and water according to a mass ratio, wherein the cement content is 5%, 10% and 15%, and the water-solid ratio is 0.2;

For example, Portland cement PO42.5 may be used as cement.

Filling parameter setting: the filling height is converted according to the proportional scale model (at a scale of 1:100 or 1:200). The equivalent height is set to 100 m, and the model heights are set to 0.2 m, 0.4 m, and 1.0 m.

Cementing conditions: dry tailings with 0% cement, and cemented tailings with cement contents of 5%, 10%, and 15%.

Dry tailings filling: the conveying assembly 200 is started, and a flow rate of 30 kg/min is set. The tailings are uniformly spread at the bottom of the open pit model 101, with each layer having a thickness of 10 cm and a tolerance of ±1 cm.

After each layer is filled, the pressure plate 320 is started, with a frequency of 50 Hz and an amplitude of 2 mm. A pressure of 0.5 MPa is applied, and compaction is repeated three times to ensure that the compaction degree is greater than or equal to 90%.

Compaction degree calculation formula:

$$C = \frac{{ρ}_{d}}{{ρ}_{max}} \times 100 \% ,$$

where C is the compaction degree (%), ρ_{d} is the measured dry density (g/cm³), and ρₘₐₓ is the maximum dry density (g/cm³).

S300: a vertical load is applied to the filling model to obtain a compacted backfill model.

Static load: a vertical static load is applied by the hydraulic cylinder 310 at a rate of 0.1 MPa/min until the pressure corresponding to the equivalent filling height is reached.

S400: the first mining body 141 is removed and the removed part is filled with the filling body, and thereafter the second mining body 142 is removed; if the boundary pillar model 130 does not fail after mining, a static load is applied to the backfill model at a rate of 0.1 MPa/min until the boundary pillar model 130 fails; and simultaneously, the historical data of the boundary pillar model 130 is acquired and preprocessed to obtain processed data;

Step S400 is performed to simulate a mining disturbance. During the test, the strain response and acoustic emission signal of the boundary pillar model 130 are continuously recorded. Firstly, one-step room mining is simulated, and the first mining bodies 141 are excavated one by one. After complete excavation, all void positions are filled one by one with a simulated filling body. Secondly, two-step room mining is simulated, and the second mining bodies 142 are excavated one by one. After complete excavation, if the boundary pillar model 130 does not fail, a static load is applied at a rate of 0.1 MPa/min until the boundary pillar fails.

The internal strain distribution of the boundary pillar model 130 is monitored in real time by the fiber grating sensor 410 at a sampling frequency of 1 kHz.

The event count rate and energy accumulation value of the acoustic emission signal are captured by the acoustic emission probes 430, and high-frequency noise is eliminated by a wavelet threshold denoising method.

The surface temperature distribution of the boundary pillar model 130 is collected by the thermal infrared cameras 440 at a frequency of 5 Hz, a heat map is generated, and the temperature data are smoothed by a moving average filter.

Sensor calibration, fiber grating sensor 410: a known displacement is applied using a strain calibration frame with an accuracy of 1 μm, and the strain sensitivity coefficient is calibrated.

Sensor calibration, fiber grating sensor 410: a known displacement is applied using the strain calibration frame with an accuracy of 1 μm, and the strain sensitivity coefficient

$${K}_{ε} = \frac{Δλ}{{λ}_{0} Δε}$$

is calibrated, where K_{ε} is the strain sensitivity coefficient (pm/με), Δλ is the wavelength variation (pm), λ₀ is the initial central wavelength (nm), and Δε is the strain variation (με).

Acoustic emission probe 430: a pencil lead break method, Hsu-Nielsen source, is used, the sensitivity is calibrated, and the frequency response range is ensured to be from 20 kHz to 1 MHz.

Pre-embedded sensors: a hole with a diameter of 5 mm is drilled inside the boundary pillar model 130, and the fiber grating sensor 410 is implanted therein. The hole is sealed with epoxy resin to ensure that the strain transfer efficiency is greater than or equal to 95%.

Temperature field monitoring: the surface temperature distribution of the boundary pillar model 130 is collected by the thermal infrared cameras 440 at a frequency of 5 Hz, and a heat map with a resolution of 640×480 is generated.

Wavelet threshold denoising: the acoustic emission signal is decomposed using 5-layer wavelet decomposition with the db4 basis function, and soft threshold processing is used to eliminate high-frequency noise.

Moving average filtering: a moving average filter with a window length of 10 is applied to the temperature data. The filtering formula is as follows:

$${T}_{filtered} = \frac{1}{N} {\sum }_{i = 1}^{N} {T}_{i} ,$$

where T_{filtered} is the filtered temperature value (° C.), Tᵢ is the original temperature data point, and N is the size of the sliding window.

Feature extraction: strain characteristics are extracted. The mean value of strain is calculated:

$${μ}_{ε} = \frac{1}{n} {\sum }_{i = 1}^{n} {ε}_{i} ,$$

and the variance is calculated as

$${σ}_{ε}^{2} = \frac{1}{n} {\sum }_{i = 1}^{n} {( {ε}_{i} - {μ}_{ε} )}^{2} ;$$

where μ_{ε} is the mean value of the strain, σ_{ε} is the variance of the strain, n is the total number of strain data points, and εᵢ is the i^{th} strain data point.

Acoustic emission frequency domain analysis: the proportion of dominant frequency energy is extracted by Fast Fourier Transform (FFT) using the formula

$$R = \frac{{E}_{m a i n}}{{E}_{t o t a l}} \times 100 \% ,$$

where Eₘₐᵢₙ is the dominant frequency band energy, and Eₜₒₜₐₗ is the total energy.

S500: the processed data is input into the hybrid deep learning model to obtain the trained hybrid deep learning model.

The strain, acoustic emission, and temperature data are input into the hybrid deep learning model and the LSTM_CNN fusion framework. A pillar safety factor (SF) is calculated in real time, and the loading conditions are adjusted dynamically according to an early warning mechanism.

Model architecture design: in the LSTM branch, the input is a strain and acoustic emission sequence with a time window length of 60 s and a sampling interval of 0.1 s, including a total of 600 data points. The LSTM branch is provided with a 3-layer long short-term memory (LSTM) unit and a hidden layer node of 128, and a time-dependent feature vector is output. In the convolutional neural network (CNN) branch, a crack image of 224×224 pixels and a thermal temperature map are input. A pre-trained ResNet_50 is used to extract spatial features, and fusion layers are applied. The outputs of the LSTM and CNN branches are spliced and mapped to the SF through a fully connected layer with nodes 256→128→1.

Model training: the loss function used is Huber loss, which is given by the following formula:

$${L}_{δ} ( y , \hat{y} ) = \{ \begin{matrix}\frac{1}{2} {( y - \hat{y} )}^{2} & if \vert y - \hat{y} \vert \leq δ \\ δ \vert y - \hat{y} \vert - \frac{1}{2} {δ}^{2} & otherwise\end{matrix} ;$$

Optimizer: the Adam optimizer is used, with a learning rate of 1e-4, a batch size of 32, and a training cycle of 100.

where y is the true safety factor, ŷ is the predicted safety factor, and δ is the threshold parameter, which is set to 1.0.

S600: the real-time data are input into the trained hybrid deep learning model to obtain the safety factor of the pillar. The real-time data include strain, acoustic emission, and temperature data. When the safety factor of the pillar is greater than or equal to 0.8 and less than 1.0, the loading rate is reduced to 0.05 MPa/min. When the safety factor of the pillar is greater than or equal to 0.5 and less than 0.8, the application of static load to the backfill model is suspended. When the safety factor of the pillar is less than 0.5, the application of static load to the backfill model is stopped, the alarm is triggered, and the experimental data are saved.

Logic for calculating the safety factor (SF):

$$SF = \frac{critical load}{current load} .$$

The critical load is fitted from historical test data.

Three-level warning mechanism: Level I warning: when 0.8≤SF<1.0, the loading rate is reduced to 0.05 MPa/min, and the data are manually checked for abnormality.

Level II warning: when 0.5≤SF<0.8, the loading is suspended.

Level III warning: when SF<0.5, the pressure control assembly 300 is immediately cut off, an audible and visual alarm is triggered, and the experimental data are saved.

Failure mode analysis and critical parameter calculation are performed. The specific steps are as follows:

Failure mode determination: tensile failure: the crack propagates symmetrically along the centerline of the boundary pillar model 130, the acoustic emission events are concentrated in the middle and upper part, with the coordinate x being greater than 0.5 L, where Lis the pillar height.

Shear failure: when the cracks cross obliquely at an angle of 45°-60°, and the cumulative value of acoustic emission energy in the bottom area accounts for greater than or equal to 60%.

Calculation of critical parameters: safe filling height: the experimental data are fitted using logistic regression, with the formula:

$${H}_{s a f e} = \frac{{H}_{max}}{1 + {e}^{- k ( {f}_{c} - {f}_{0} )}} .$$

Cementing strength threshold: the Weibull distribution is used to analyze the failure probability, and the minimum cement content with a 95% confidence interval is determined.

where H_{safe} is the safe filling height (m), Hₘₐₓ is the maximum filling height (m), k is the material attenuation coefficient, f_{c} is the compressive strength of cement (MPa), and f₀ is the base strength (MPa).

In the above description of the present disclosure, it is to be noted that the terms ‘first’ and ‘second’ are used for descriptive purposes only and are not to be understood as signifying or implying relative importance or as implying the number of technical features indicated. Accordingly, features designated as ‘first’ or ‘second’ may explicitly or implicitly include one or more such features. In the description of the present disclosure, the term ‘multiple’ means two or more, unless otherwise explicitly specified.

In the present disclosure, unless otherwise expressly provided or defined, terms such as ‘mounted’, ‘connected’, ‘connection’ and ‘fixed’ are to be understood in a broad sense, e.g. as a fixed connection, as a detachable connection or as an integral connection; may be a mechanical connection or an electrical connection; it can be directly connected or indirectly connected through an intermediate medium, and they may refer to internal communication between two components or an interactive relationship between two components. A person skilled in the art will be able to understand the specific meaning of the above terms in the context of the present disclosure based on the specific circumstances.

In the present disclosure, unless otherwise expressly specified and defined, a first feature being ‘on’ or ‘under’ a second feature may mean that the first and second features are in direct contact, or that the first and second features are in indirect contact via an intermediate medium. Moreover, a first feature being ‘above,’ ‘over,’ or ‘on top of’ a second feature may mean that the first feature is directly above or obliquely above the second feature, or merely that the first feature has a higher horizontal elevation than the second feature. A first feature being ‘below,’ ‘beneath,’ or ‘underneath’ a second feature may mean that the first feature is directly below or obliquely below the second feature, or merely that the first feature has a lower horizontal elevation than the second feature.

Throughout the description of this specification, the terms ‘one embodiment,’ ‘some embodiments,’ ‘an embodiment,’ ‘an example,’ ‘a specific example,’ or ‘some examples,’ and the like, mean that a particular feature, structure, material, or characteristic described in connection with the embodiment or example is included in at least one embodiment or example of the present disclosure. In this specification, the schematic depictions of the above terms are not necessarily directed to the same embodiment or example. Furthermore, the particular features, structures, materials, or characteristics described may be combined in any suitable manner in any one or more embodiments or examples. Additionally, combinations and subcombinations of the various embodiments or examples, as well as combinations and subcombinations of the features of the various embodiments or examples described in this specification, may be made by one skilled in the art without departing from the spirit and scope of the present disclosure.

Although embodiments of the present disclosure have been illustrated and described above, it should be understood that these embodiments are merely illustrative and are not to be understood as limiting the scope of the present disclosure; those skilled in the art may make alterations, modifications, substitutions, and variations to the aforementioned embodiments within the scope of the present disclosure.

What is claimed is:

1. A device for testing boundary pillar failure induced by tailings backfill in an open pit, comprising:
a test assembly (100);
a conveying assembly (200), connected to the test assembly (100) and configured to uniformly feed tailing into the test assembly (100);
a pressure control assembly (300), connected to the test assembly (100) and configured to control pressure in the test assembly (100); and
a monitoring assembly (400), disposed on the test assembly (100) and configured to monitor data information within the test assembly (100) when internal pressure of the test assembly (100) is changed;
wherein the test assembly (100) comprises:
a test box (110);
a test plate (120), arranged in an interior of the test box (110), and configured to form a funnel-shaped open pit model (101) in the test box (110);
a boundary pillar model (130), arranged in the test box (110) and positioned at a bottom of the open pit model (101); and
a mining body component model (140), arranged in the test box (110) and in contact with a bottom of the boundary pillar model (130), and is configured to support the boundary pillar model (130);
wherein the conveying assembly (200) comprises:
a protective housing (210), arranged on the test box (110) and formed at a bottom thereof with a plurality of discharge openings (201) in communication with the open pit model (101);
a drive shaft (220), coaxially and rotatably mounted inside the protective housing (210);
a blocking plate (230), fixedly arranged on the drive shaft (220) in an alternating manner, and the blocking plate (230) blocks the discharge openings (201) when the blocking plate (230) is in contact with the discharge openings (201);
a material storage assembly (240), in communication with the protective housing (210), and configured to convey tailings into the protective housing (210); and
a vibration component (250), arranged on an outer wall of the test box (110), and configured to uniformly spread the tailings within the open pit model (101).

2. The device for testing boundary pillar failure induced by tailings backfill in an open pit according to claim 1, wherein the mining body component model (140) comprises:
a plurality of first mining bodies (141), arranged in parallel in the test box (110) and in contact with a bottom of the boundary pillar model (130);
second mining bodies (142), arranged between the adjacent first mining bodies (141) and in contact with a bottom of the boundary pillar model (130).

3. The device for testing boundary pillar failure induced by tailings backfill in an open pit according to claim 1, wherein the material storage assembly (240) comprises:
a storage bin (241), configured to store the tailings;
a delivery pipe (242), in communication between the storage bin (241) and the protective housing (210); and
an auger (243), arranged in the delivery pipe (242) and configured to convey tailings.

4. The device for testing boundary pillar failure induced by tailings backfill in an open pit according to claim 1, wherein the monitoring assembly (400) comprises:
a fibre grating sensor (410), arranged in an interior of the boundary pillar model (130);
a pressure sensor (420), arranged at a bottom of the mining body component model (140);
a plurality of acoustic emission probes (430), arranged at one side of the test box (110) at equidistant intervals; and
thermal infrared cameras (440), arranged on other side of the test box (110) at equidistant intervals.

5. The device for testing boundary pillar failure induced by tailings backfill in an open pit according to claim 1, wherein the pressure control assembly (300) comprises:
a hydraulic cylinder (310); and
a pressure plate (320), arranged on a top of the test box (110), connected to an output end of the hydraulic cylinder (310), and configured to vary pressure in the open pit model (101).

6. A method for testing boundary pillar failure induced by tailings backfill in an open pit, employing the device for testing boundary pillar failure induced by tailings backfill in an open pit according to claim 1:
making the mining body component model (140), the boundary pillar model (130), and the open pit model (101) successively arranged, wherein the mining body component model (140) comprises the first mining bodies (141) and the second mining bodies (142) arranged adjacent to each other;
uniformly spreading and compacting tailings in the open pit model (101) with a compaction degree of greater than or equal to 90% to form a filling model;
applying a vertical load to the filling model to obtain a compacted backfill model;
removing the first mining body (141) and filling the removed part with a filling body, and thereafter removing the second mining body (142), if the boundary pillar model (130) does not fail after mining, applying a static load to the backfill model at a rate of 0.1 MPa/min until the boundary pillar model (130) fails; and simultaneously, acquiring historical data of the boundary pillar model (130) and preprocessing the historical data to obtain processed data;
inputting the processed data into a hybrid deep learning model to obtain a trained hybrid deep learning model;
inputting real-time data into the trained hybrid deep learning model to obtain a safety factor of the pillar;
when the safety factor of the pillar is greater than or equal to 0.8 and less than 1.0, reducing a loading rate to 0.05 MPa/min;
when the safety factor of the pillar is greater than or equal to 0.5 and less than 0.8, suspending the static load applied to the backfill model; and
when the safety factor of the pillar is less than 0.5, stopping the static load applied to the backfill model, triggering an alarm, and saving experimental data.

7. The method for testing boundary pillar failure induced by tailings backfill in an open pit according to claim 6, wherein steps of making the open pit model (101) and the boundary pillar model (130) further comprise:
mixing barite powder, river sand, gypsum, and water in a ratio of 1:4:1.25:1.25, casting the mixture to form the open pit model (101) at a scale of 1:100 or 1:200, and curing the model for 7 days;
mixing barite powder, river sand, gypsum, and water in a ratio of 1:3.75:1.5:1.25 to form the boundary pillar model (130), curing the model for 7 days, and then embedding the model into the open pit model (101) at a predetermined position.

8. The method for testing boundary pillar failure induced by tailings backfill in an open pit according to claim 7, wherein the step of making the filling model further comprises:
mixing tailings, cement, and water according to a mass ratio, wherein a cement content is selected from 5%, 10%, and 15%, a water-to-solid ratio is 0.2, and the tailings have a particle diameter of less than 2 mm.
